(19) United States
(12) Patent Application Publication
(10) Pub. No.: US 2006/0191544 A1
(43) Pub. Date: Aug. 31, 2006

(54) Instrumented thoracic model

(76) Inventors: Kirth E. Simmonds, Clinton, MD (US); Richard K. Everett, Alexandria, VA (US); Peter Matic, Alexandria, VA (US); Alan Chun Ning Leung, Burke, VA (US)

Correspondence Address: NAVAL RESEARCH LABORATORY;ASSOCIATE COUNSEL (PATENTS), CODE 1008.2, 4555 OVERLOOK AVENUE, S.W., WASHINGTON, DC, 20375-5320, US

(21) Appl. No.: 11/365,952

(22) Filed: Feb. 28, 2006

Related U.S. Application Data
(60) Provisional application No. 60/656,385, filed on Feb. 28, 2005.

(51) Int. Cl. C12Q 1/00; A61B 5/00; A61K 36/54

(52) U.S. Cl. 128/898; 435/4; 600/300; 424/739

(57) ABSTRACT

An nstrumented thoracic model for measuring the effects of impacts is provided. The model has simulated skeletal components, simulated tissue, simulated internal organs, and sensors that are optimally placed in the simulated tissue and organs, said simulated organs and said simulated skeletal components. Simulated human tissue is made of a modified ballistic gelatin, comprising ordinance gelatin, chilled water and an antimicrobial agent in a desired volume or weight percentage. The resulting mixture is then poured into a container or mold having the desired tissue shape, and then chilled until the mixture has set. Simulated lung tissue is made of the modified ballistic gelatin, but also incorporates nicrospheres to approximate the density and modality of the lungs. The sensors of the thoracic model are optimally placed using primary component analysis

### CROSS-REFERENCE TO RELATED APPLICATIONS

This application claims the benefit of U.S. Provisional Application No. 60/656,385, filed on Feb. 28, 2005, the entirety of which is incorporated herein by reference.

### BACKGROUND OF THE INVENTION

On the battlefield, ground forces are at great risk from nonpenetrating blunt trauma forces and blast dynamics that may cause injury to vital organs. The biodynamics describing the mechanisms of these injuries is not well understood. Tissue surrogate materials that simulate the mechanical and acoustical properties of biological tissues assist in understanding the biodynamics. Tissue surrogate materials are assembled into an experimental test system of the human thorax for assessing blunt forces and blast dynamics. With accurate surrogate systems, the manner in which the blast wave interacts with the thorax and any protective equipment worn can be determined, as well as how distinct pressure time histories and strain rates produce loads on tissues. Beyond the obvious military applications, potential civilian applications include understanding automobile crash injuries, nonlethal projectiles used by law enforcement personnel, the performance of blast-resistant office structures for protection against terrorist attacks, and blasts in mines, refineries, and grain elevators.

Homogeneous blocks of ballistic gelatin combined with high-speed photography, videography and post-test analysis have been used for many years to assess wound or terminal ballistics. Correlation of bullet tracks and gel damage to wounds has always been problematic. Homogeneous clay blocks have also been used to assess the performance of ballistic armor, but no clear relation to human performance has been established.

Other methods use instrumented cylindrical and elliptical shapes that are made of metals and combination of composite materials that include metals. These methods measure blast dynamics and simulations and correlations are used to estimate propagations around the thorax. Because these devices are not anatomically correct and do not have the close mechanical properties of the human thorax, the transfer of impact energy from behind body armor is not accurately measured for blast and ballistic impacts. The major limitation of these devices is the lack of measuring any internal failure mechanism associated with the visco-elastic properties of the various tissues and organs. Other methods involve the use of live animals and human cadavers, which have moral and ethical issues that limit the research on injury mechanisms.

Biermann, et al, U.S. Pat. No. 6,769,286 discloses an instrumented torso model that simulates anatomical features and measures the effects on a body caused by various types of impacts. Biermann uses tissue simulants known in the art, which do not adequately simulate the response of human tissue to impact. Additionally, Biermann does not discuss the procedure for determining proper placement of sensors in order to obtain impact test results that best simulate an impact on human tissue.

Therefore, there is a need for a system and method that can reproducibly simulate the complex wave propagation environment found in the human thorax. There is a further need for a system and method to measure the internal response from blasts, impacts and overpressures in a manner that can be directly correlated to animal and cadaver models. These and other needs are met by the present invention. The present invention comprises an instrumented thoracic model having simulated skeletal components, including, but not limited to, anatomically correct spine, rib cage, and sternum; simulated tissue surrounding said simulated skeletal component; simulated organs, including but not limited to, a heart and two lungs, enclosed in said simulated tissue; and sensors, optimally placed in said simulated tissue, said simulated organs and said simulated skeletal components for measuring the effects of impacts on said thoracic model.

### BRIEF SUMMARY OF THE INVENTION

Simulated human tissue is made of a modified ballistic gelatin, comprising ordinance gelatin, chilled water and an antimicrobial agent in a desired volume or weight percentage that is hydrated for a period, then gradually heated while mixing until clear. The resulting mixture is then poured into a container or mold having the desired tissue shape, then chilled until the mixture has set. Simulated lung tissue is made of the modified ballistic gelatin, but also incorporates microspheres to approximate the density and modality of the lungs. The sensors of the thoracic model are placed using primary component analysis, PCA. The PCA analysis is applied to the heart, lungs and spine by taking the normalized displacements of all the primary modes; calculating the principal components from the normalized displacements; selecting only the principal components which have a significant influence on the system; using the major principal components on the normalized displacement data; reevaluating the reduced data set for patterns, relationships; and grouping/clustering for placement of the sensors.

### BRIEF DESCRIPTION OF THE DRAWINGS

FIG. 1 is a view of the thoracic surrogate model;

FIG. 2 shows the stress strain relationship measured for the surrogate lung material, actual human lung tissue, and ordnance gelatin.

FIG. 3 shows the anatomically correct lungs and the heart made from standard molds.

FIG. 4 shows the approximate placement of the sensors.

FIG. 5 is a simple ordnance gel block with a cylindrical “lung” inside;

FIG. 6 is a schematic of the test setup and corresponding finite element model;

FIG. 7 shows the input uniaxial compression and plane strain compression experimental data used in the simulations for the ordnance gelatin.

FIG. 8 shows the measured displacements histories for the experimental cases at impact energies of 2, 3, 5, and 10 Joules for the 1045 gram ball for the accelerometer placed at the center of the block

FIG. 9a and 9b show the predicted displacement history from the finite element and experimental models' response for the 1045 gram ball at the center of the block with impact energies of 3 and 5 Joules, respectively

FIG. 10 shows the predicted displacement responses of the specimen using 1045 and 1390 gram balls with an impact energy of 5 Joules at the center of the specimen (FIG. 10a) and at 51 mm from the impact surface (FIG. 10b)

FIG. 11 a-d shows Pressure contour plots of the 10 Joule, 1390 gram ball drop case for times of 0.027, 0.066, 0.076, and 0.081 seconds, respectively.

FIG. 12 shows a steady-state modal analysis of the thoracic surrogate model.

### DETAILED DESCRIPTION OF THE INVENTION

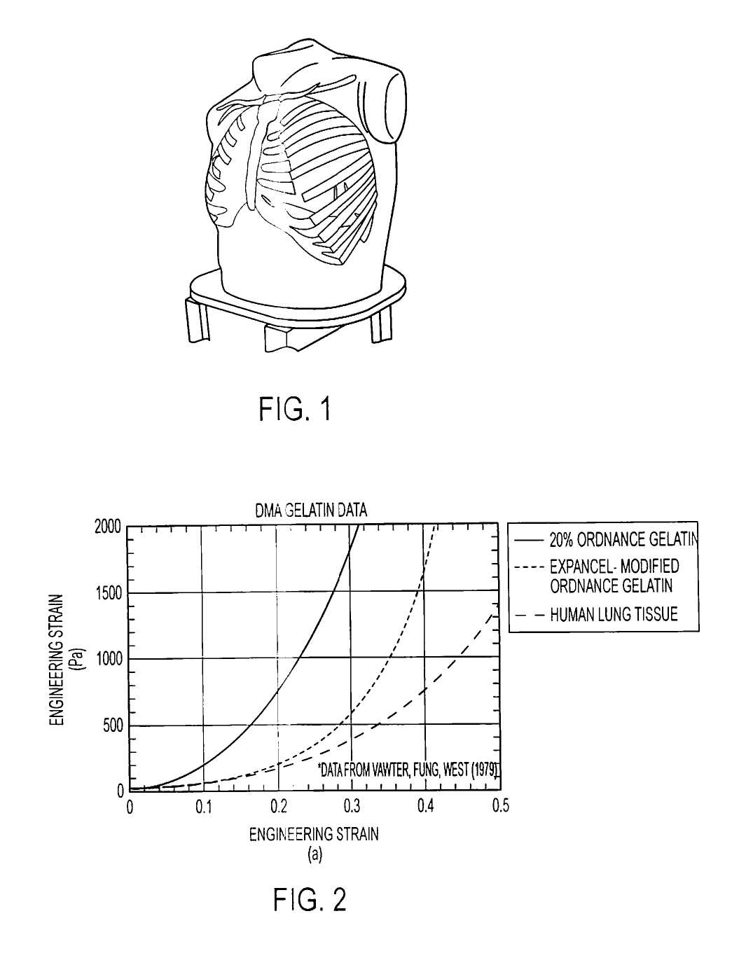

A thoracic surrogate model comprised of a spine and rib cage from a plastic skeleton reproduction surrounded by tissue simulant with high fidelity lungs and heart organs is described. A thoracic surrogate model is shown in FIG. 1. The thoracic surrogate model comprises geometrically-realistic surrogate organs comprised of tissue simulants of various formulations with engineered material properties. The organs are encased in a realistic skeletal system, which is available commercially. The thoracic surrogate model is internally instrumented with pressure sensors and accelerometers attached to the skeletal elements and within the organs and interfaces. Each element and the construction of the thoracic surrogate model will be described in detail.

In order to achieve accurate thoracic modeling, several factors have to be addressed. Accurate geometry in the thoracic model is important. In general, the model should be as simple as possible to aid computation but still complicated enough to reproduce the desired phenomena. Simpler models can provide guidance in constructing more complicated models. For example, the analysis of the response of cylindrical lungs was compared to gelatin block experiments with controlled impacts. FEM analysis also confirms good correlation. FEM analysis of ball-drop experiments, which provide controlled impact conditions, where only human tissue properties, as gathered from the literature, have been used as input to the FE models match the experimental measurements very well.

Blast phenomena are extremely complicated. The body has many density discontinuities which affect the way sounds propagate. Bouncing waves may cause damage a long distance away from where they entered, or only in combination with other bouncing waves. For example, waves can run around the rib cage, into the spine, and perhaps even travel to the brain. Very simple geometrical structures lack the boundaries and interfaces which generate the wave “bounces.” Therefore the anthropomorphic nature of the present thoracic surrogate is highly important. The present model incorporates enough detail to get “the bounces” which cause damage/injury, however it is not too complicated, so the results can be modeled and interpreted.

Additionally, the model simulant material has to respond like real human tissue. Organs comprised of metals would not behave like human tissue, regardless of their shape. The determination of how the human tissue behaves is not simple. Human tissues can change rapidly in ex-sanguineous environments and can vary with ex vivo temperature and time. However, the features of human tissue that control wave propagation, deformation, and ultimately failure are deemed important. Materials failure may be different from functional failure. The ability to know and control the various factors such as density and stiffness control wave speed; initial modulus, yield stress, secondary modulus, hardening rate, and ultimate tensile strain control deformation in these viscoelastic materials is important. Linear elastic models sometimes used in FEM can be quite misleading as they predict structures to be much too stiff in response to loads. Generally, modulus, yield stress, secondary modulus, hardening rate, and ultimate tensile strain are very sensitive to the speed that they are deformed at; that is they are strain rate sensitive. While it is possible to match the room temperature low-strain-rate properties, very little is known about the high-strain rate regime.

Various formulations of ballistic gelatin and EXPANCEL® microspheres were studied in order to mimic human tissue properties. Ultrasonic wave speed as “high-strain-rate” was studied. DMA (dynamic mechanical analysis) testing was used for the lower speed deformation. The Expancel microspheres generally reduce the density and modulus while other fillers such as silica or alumina can be used to increase density and modulus. The property variations may be estimated from composite theory. The match to human tissue properties is not perfect but is good at small strains, thus the model responds fairly accurately for small strains. Those skilled in the art would understand that other additives to ballistic gelatin could also provide similar results. For example, glass-shell microspheres, cenospheres, and organic micro- and nano-spheres such as liposheres could be used for reducing the density and modulus of the simulated tissue material.

Additionally, sensors are placed within the thoracic surrogate to determine when waves, which are recorded as pressure changes, pass certain sensor locations, and to determine when certain features move, from the accelerometers. Obviously, the model behavior cannot be measured at each and every point. Placement of some sensors is easily determined from observed injury statistics. Determining the placement of other sensors can be less obvious, as in the case of the inside of the lung. The optimal sensor positions are those that provide the best predictions using the FE models. Principle Component Analysis (PCA) analysis was used to determine the optimum placement of the sensors in order to take the measurements needed to determine the behavior of the thoracic surrogate.

The anthropomorphic torso model is subjected to some unknown stimulus such as an impact. The pressure sensors and accelerometers are measured as a function of time after impact. Blast or impact waves can vary depending on the type of explosive, the amount, the range to the detonation point, and the presence and shape of reflecting surfaces. The simplest case is a free-field explosion that produces a pressure pulse known as a Friedlander or ideal blast wave. Most real cases are more complex. The thoracic surrogate model response depends on the orientation of the model, for example anterior to posterior as well as upright to prone, and how the model is protected or clothed, for example with armor or a shirt. The model must have been calibrated in some manner for the measurements to correlate to human tissue response. The measurements for an uncalibrated model will demonstrate how the model responds but not how that response relates to how a human body would respond. Models that do not have simulated materials with human tissue like properties cannot be expected to have a human like response. For example, some models use a polyurethane material as tissues simulant. This material is tougher than human tissue and does not cavitate in the manner of human tissue during ballistic penetration. In the case of thoracic surrogate, the similitude has been built in and tested in various controlled situations with calibrated stimuli.

The simulated tissue used in the thoracic surrogate is comprised of Ordinance TYPE 250A Gelatin, distilled water or RO (Reverse Osmosis) water chilled to 7-10° C., Cinnamon 100% pure leaf oil (Aura Cacia—cinnamomum zeylancium), and, for the lung tissue, EXPANCEL® DE (Akzo Nobel Product 091 DE 80 d 30). EXPANCEL DE has a particle size of approximately 60-90 micrometers. Cinnamon oil acts as an antimicrobial agent. Those skilled in the art would understand that other antimicrobial agents could be utilized.

Generalized simulated tissue (20% volume fraction of gelatin) was formed by combining a volume percentage or weight percentage of gelatin in water, stirring while pouring. For example, 200 ml of gelatin to 800 ml of water results in a 20% volume fraction solution. Typically, glass beakers were used to ensure no undesired chemical reactions occurred, for example, chemical reactions between stainless steel and gelatin. Additionally, a hotplate with magnetic stirrers and thermocouples with feedback control were well suited for the heating step. A thermocouple or temperature measuring device should be approximately 6.55 mm above the bottom of the beaker to monitor the temperature of the mixture.

For example, 2800 ml of chilled water was combined with 700 ml of the Gelatin while stirring. Approximately 10 drops of the cinnamon oil was added to that mixture. The mixture was stirred and allowed to hydrate in cold water (7-10° C.) for about 2.5 hours to overnight. Preferably, the mixture is allowed to hydrate at least 5 hours.

After hydration, the mixture is heated while stirring, starting from approximately room temperature increasing by increments of 20° F. up to about 140° F. (60° C.) over about 4 hours, until mixture is clear, indicating all hydrated gelatin crystal have liquefied, and displays no turbidity. Approximately 5 drops cinnamon oil is added to this mixture, which is then stirred at a stir speed of approximately 500 rpms. Temperatures exceeding 159° F. (71° C.) are not recommended, as burning or other damage to the desired gelatin properties may occur. The stirring speeds should be such that there is no formation of bubbles created by a vortex pulling in air from the top surface of the solution.

The final mixture is poured into a mold or container with the desired shape without causing air formation on the surface of the mold or container. A plastic rod can be placed at the lip of the container for controlling the flow of the mixture. The filled mold or container is chilled at a temperature from about 7° C. to about 10° C. for approximately 12-24 hours, depending on the volume of the mixture.

The simulant tissue material for the simulated lung tissue is created as the general simulant tissue; however, the heating step is conducted in a fume hood with fan speed set at approximately 60%. After the heating step, approximately 0.42 grams of the EXPANCEL® DE microspheres are added to the 3500 ml of liquid gelatin (2800 ml of water/700 ml gelatin/cinnamon oil, above). The EXPANCEL DE microspheres can be added in a range from about 0.30 grams to about 0.60 grams per 3500 ml. Preferably, the stirring mechanism is set at the highest available speed, preferably creating a vortex in the mixture. The final mixture is poured into a mold or container with the desired shape without causing air formation on the surface of the mold or container. A plastic rod can be placed at the lip of the container for controlling the flow of the mixture. The filled mold or container is chilled at a temperature from about 7° C. to about 10° C. for approximately 12-24 hours, depending on the volume of the mixture. Molds and containers can be any known in the art. Preferably, plastic molds, such as Nalgene, or other polymers that do not attract bubble formations along the mold or container are used.

The resulting tissue simulant is viscoelastic, therefore the injury mechanism can be potentially quantified in terms of material properties and the possible failure mechanism associated with the injury. Injury can also be through loss of function as well as accumulated damage. The tissue simulant used is comprised of a gelatin formulation having birefringence properties, which allows photoelasticity to be used to visually observe both static and dynamic strain patterns as a function of load using high-speed videography. In the formulation lungs for the model, EXPANCEL® DE microspheres, small spherical plastic particles that contain gas, are used to create lung tissue simulants that can be adjusted to match the wide variation material properties existing in human populations. The tissue simulants used in the model allow the transfer of impact energies to be more representative of what occurs in humans.

Alternative tissue simulant materials may be used if the viscoelastic performance and mechanical properties are comparable to human tissue so that the instrumentation can measure and define blast and blunt trauma injury mechanisms. Some polymers have the feel or touch of human tissue, however the actual performance is not always adequate for simulating the tissue dynamics/physics. Gel wax candle materials has been used by others as a potential substitute for ordnance gelatin with ballistic performance in terms of penetration and cavitations. Other materials such as silicone elastomers show promise, however do not exhibit cavitations as found in ordnance gelatin. Silicone gels do exhibit some degree of failure and fracture or tearing at high strains, however.

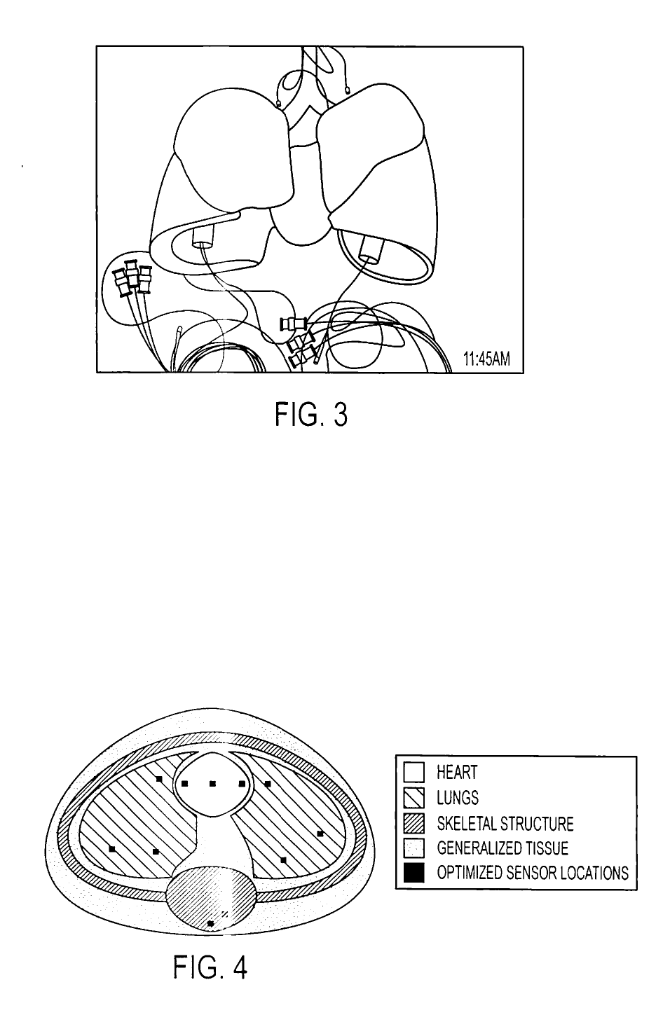

The development of materials that respond in the same manner as biological tissue, for example duplicating the acoustical and mechanical responses, is an important factor in studying the mechanisms that lend to the trauma of living organisms. By using the modified ordnance gelatin, tissue simulant formulations have been developed that have the necessary acoustical and mechanical bulk properties for the human lungs and heart. FIG. 2 shows the stress strain relationship measured for the surrogate lung material, actual human lung tissue, and ordnance gelatin. Molds of standard lungs and the heart are used to make anatomically correct organs, shown in FIG. 3, used in the thoracic surrogate model.

The anatomically shaped organs include, but not limited to, a heart and two lungs, are instrumented with pressure sensors and 3-axis accelerometers. One skilled in the art would understand that other sensors could also be used. Additional small-scale features in the lungs could include bronchial passages, tailored porosity to represent alveoli structures and the inclusion of horizontal and oblique fissures. Additional features of the heart could include coronary arteries. Other organs can be adaptively simulated, including, but not limited to the brain, including the left and right lobes, the cerebellum, the brain stem, the spinal column, the subarachnoid space, and the cerebral spinal fluid; air filled structures such as the trachea, esophagus, stomach, large and small intestines; solid organs such as the liver, kidneys and pancreas; muscle groups such as the diaphragm; and other tissues such as the adipose layer under and around the chest, and the pleura.

The skeletal system of the thoracic surrogate model is available commercially. Typically, a spine, sternum, and rib cage encase the tissue simulant material. Additionally, the clavicle, scapula and pelvis is included. Costal cartilage is modeled using a plastic material. Additionally, the skull and extremities could be included.

At least one pressure sensor and at least one 3-axis accelerometer are attached to the spine. At least one pressure sensor and at least one 3-axis accelerometer are attached to the sternum. Data channels are provided are provided, as is a means for measuring acceleration and pressure in response to an applied force on the thoracic surrogate model. These measurements can be used to calculate velocity, displacement, and effective (RMS) pressure. Those skilled in the art would understand that the system could be easily adapted to include other sensors and measurements.

The placement of the sensors is determined using principal component analysis (PCA). The greatest damage to a structure such as the thoracic surrogate occurs for any type of impact loading if the frequency content of this system is close to the its natural frequency.

A finite element modal analysis can thus identify the mode shapes and frequencies where the greatest damage of the model will occur. However, the size of the finite element model can make the determination of the important variables as well as the relationship between the different variables difficult. Approximately 500,000 nodes exist in the thoracic model.

In general, principal component analysis (PCA) is a technique used to study complex systems by reducing the number of variables and detecting the relationship between variables. It is advantageous compared to other similar data analysis methods in the fact that it is a quick, efficient, yet simple technique. PCA was applied to determine optimal accelerometer sensor placement for the thoracic surrogate used in the low velocity impact test, shaker table/shock tube tests and blast tests. The participation factors obtained from the finite element modal analysis provide the primary (Mode 1, 2, 4, 5 and 6) resonance modes in the thoracic surrogate.

Sensors in the experimental tests were placed on a planar cross section to observe the progression of the blast wave in the thorax. The same planar cross section was taken from the computational model for the sensor optimization. PCA analysis was then applied to the heart, lungs and spine by: 1. taking the normalized displacements of all the primary modes, 2. calculating the principal components from the normalized displacements, and 3. selecting only the principal components which have a significant influence on the system, 4. using the major principal components on the normalized displacement data, 5. reevaluating the reduced data set for patterns, relationships and grouping/clustering for placement of the sensors. Sensor accuracy was approximately ±1 cm for the current work but can be reduced by increasing the number of data points. Three sensors were selected for the heart and lungs and one sensor was selected for the spine to utilize all the available data acquisition channels for the experimental tests. Only one sensor was used for the spine as its stiffness would provide less insight into the dynamic human behavior as compared to the heart and lungs. The FIG. 4 shows the approximate locations of the optimized sensors.

The accuracy of the PCA analysis to determine these sensor locations can be influenced by the interfaces that exist between the different parts (for example, heart to generalized tissue) of the thoracic surrogate used in the experimental tests. To simplify the analysis, this was not incorporated into the computational model and can influence the sensor placement if these interfaces have a significant effect.

Linear elastic tissue material properties were used in the computational thoracic model to simplify the computational analysis. If the material response were nonlinear, the computational model would need to be rerun with accurate nonlinear material models. A new PCA analysis would then need to be performed.

### EXAMPLE 1

### Finite Element Modeling

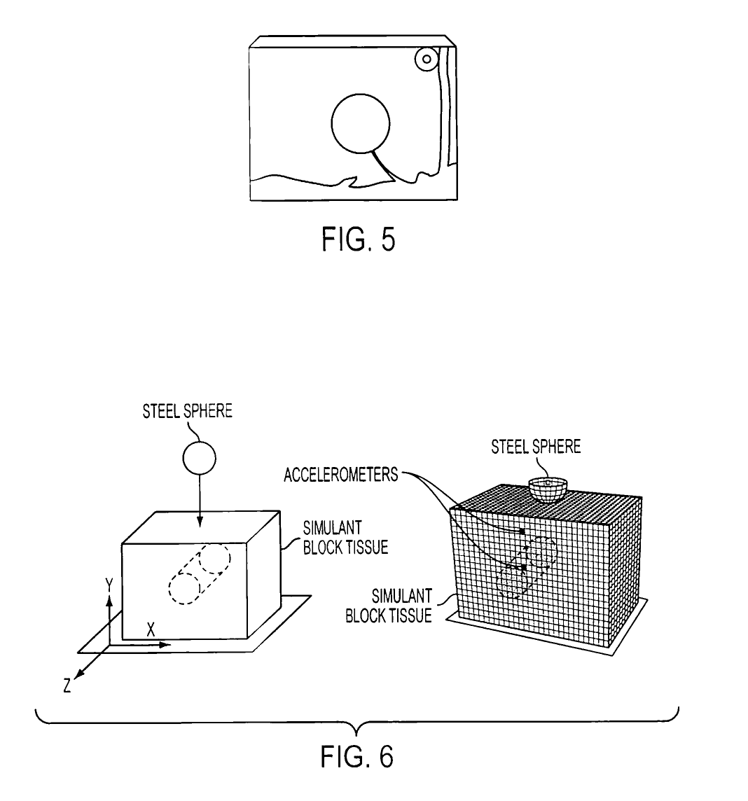

Finite element analysis studies the dynamic behavior of human tissue simulants in a relatively simple geometry under impact loading conditions. The human tissue simulants were designed to reproduce the mechanical properties of different human tissues, such as the lung and heart. Due to the inherent complexity of the thoracic surrogate model, simple tests were developed to study the mechanical response of the tissue simulants. These tests consist of blocks of human tissue simulants impacted by steel spheres of different diameters and masses at specific kinetic energy values. FIG. 5 shows an example of the simple ordnance gel block with a cylindrical simulant lung in the middle.

The experimental and finite element models consist of a rigid sphere impacting a rectangular block of the human tissue simulant situated on a table. A schematic of the test setup and corresponding finite element model is shown in FIG. 6. The dimensions of the simulant blocks were 305 mm×191 mm×216 mm. The experimental block was created by filling a rectangular mold with the desired tissue simulant of standard ordnance gelatin, leaving a cylindrical hole that was parallel to the impact surface. This cylindrical hole is backfilled with different types of simulant materials: the standard ordnance gelatin or modified ordnance gelatins represented by baseline lung tissue simulant or heart tissue simulant. This manufacturing process produces an interface between the block and embedded cylinder human tissue simulant materials that is only accounted for by a change of material property in the current finite element models. In the experiments, two accelerometers were placed in the block directly under the initial sphere impact point. The first accelerometer was located at approximately 51 mm beneath the impact surface, while the second one is located in the center of the block, approximately 121 mm beneath the surface. The bottom boundary condition consisted of a rigid flat surface, which corresponds to a table in contact with the specimen block. Both contact surfaces, the ball with the top of the specimen and the bottom of the specimen with the table, were modeled without fiction. The dimensions of the rigid flat surface were 237 mm×335 mm.

The specimen blocks were dynamically loaded by dropping different spherical ball masses to produce specific impact energies for the experimental tests. Experimentally, the selected impact energies were used to calculate the height that a specific ball was dropped. From this data, the final velocity of the ball just prior to contact with the specimen block was calculated neglecting air drag. These velocities are used as the initial conditions on a rigid hemisphere in the finite element simulations. The ball diameters, masses and impacted energies used for the computational and experimental tests are shown in Table 1.

TABLE 1
Ball diameters, masses and energies.

| | 2 Joules | | 3 Joules | | 5 Joules | | 10 Joules | |
|---|---|---|---|---|---|---|---|---|
| Experimental Tests | | | | | | | | |
| Ball Diameter | 63.5 | mm | 63.5 | mm | 63.5 | mm | 63.5 | mm |
| Ball Mass | 1045 | grams | 1045 | grams | 1045 | grams | 1045 | grams |
| Finite Element Tests | | | | | | | | |
| Ball Diameter | | | 63.5 | mm | 63.5 | mm | | |
| Ball Mass | | | 1045 | grams | 1045 | grams | | |
| Ball Diameter | | | 69.8 | mm | 69.8 | mm | | |
| Ball Mass | | | 1390 | grams | 1390 | grams | | |

These cases were selected in order to provide an understanding of how both different sphere impact energies with the same ball diameter as well as different ball diameters with the same impact energy influences the specimen's dynamic response.

ABAQUS/Explicit was used to model the impact cases. The block consisted of 32,306 C3D8R elements and 35,497 nodes. Contact pairs were defined for the rigid master surfaces, the sphere and the table, against the slave surrogate tissue block. An adaptive mesh with hourglass stiffness and an orthogonal kinetic split was used to prevent element distortion. Gravity effects were also included in the model for a more accurate response to the dynamic loading conditions.

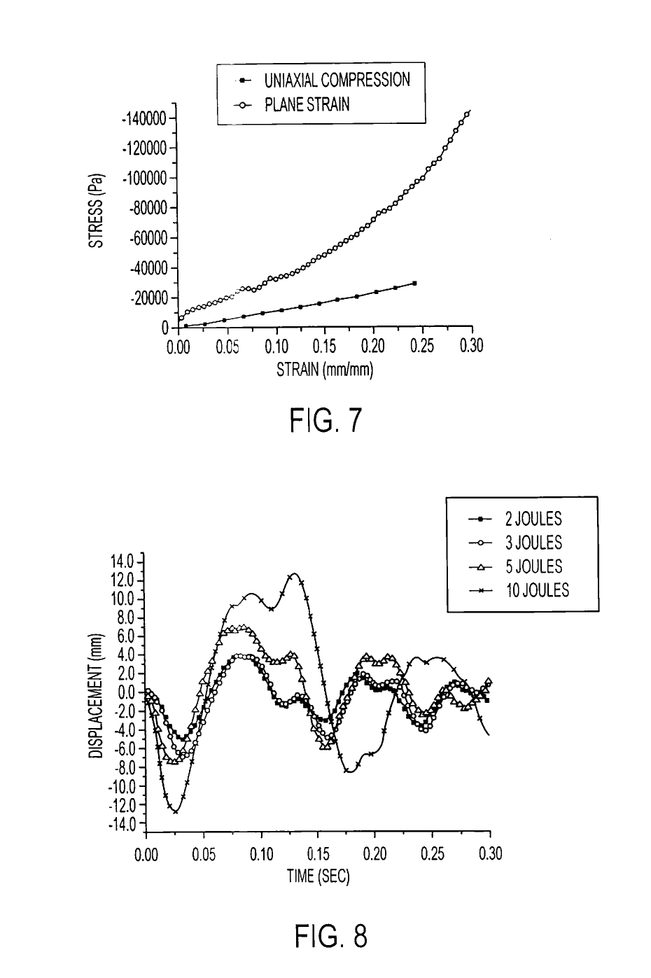

Accurate finite element predictions require the application of the appropriate constitutive models. Previous experience has shown that a hyperelastic constitutive model provides the most accurate description for the ordnance gelatin. A hyperelastic constitutive model was also selected to describe the dynamic behavior of the other two tissue simulant materials. Uniaxial and planar compression tests were performed on the different simulant materials and used as test input data. Uniaxial tension experiments were not performed due to the materials' limited tensile strengths. FIG. 7 shows the input uniaxial compression and plane strain compression experimental data used in the simulations for the ordnance gelatin. The Ogden form of the *HYPERELASTIC card was used for the strain energy potential equation with N set to 3. A linear-elastic parametric study has also been performed showing negligible influence of the Poisson's ratio on the material's response. The Poisson's ratio of the material was thus chosen to be 0.475, which is the default choice used in ABAQUS/Explicit. The density of the ordnance gelatin was 1.067 grams/cm³.

Ball drop tests on human tissue simulant blocks were performed using computational and experimental methods for different impact energies and sphere masses. The results presented below are for the case when the simulant block and the cylindrical “lung” are made solely from the ordnance gelatin base material. The displacement histories at the center of the surrogate block for the finite element and experimental models were extracted in the impact direction. FIG. 8 shows the measured displacements histories for the experimental cases at impact energies of 2, 3, 5, and 10 Joules for the 1045 gram ball for the accelerometer placed at the center of the block. As expected, the displacement magnitude increases as the impact energy is increased The 10 Joule ball drop case also exhibits higher secondary oscillations than that of the 2, 3 and 5 Joule cases. This effect may be due to different deformation mechanism activated at higher energies. Also, a definite widening of the displacement history in the 10 Joule case is observed and hypothesized to be a result of the simulant material entering the nonlinear regime.

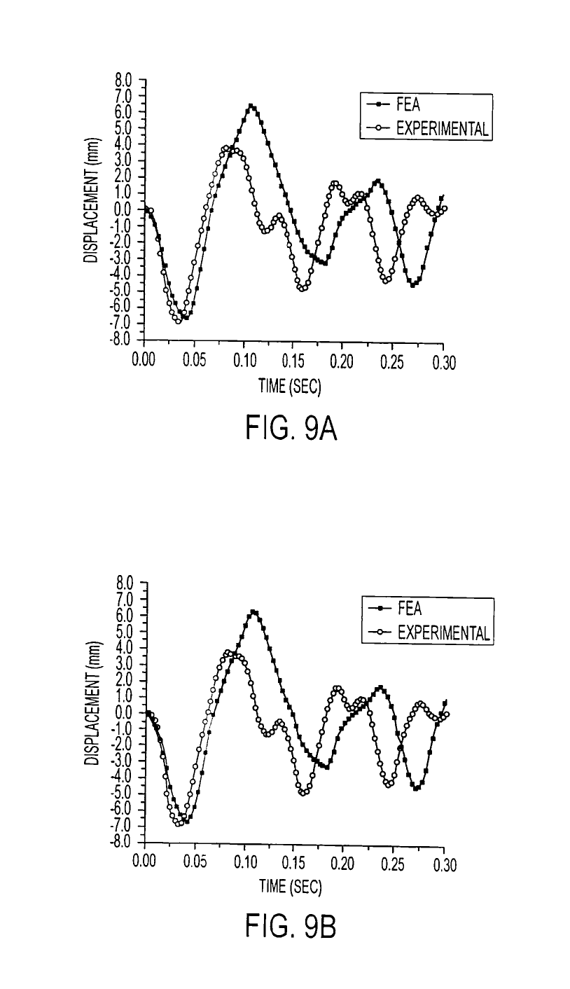

FIG. 9a and 9b show the predicted displacement history from the finite element and experimental models' response for the 1045 gram ball at the center of the block with impact energies of 3 and 5 Joules, respectively. There is relatively good agreement between the magnitudes of the predicted displacements of the finite element models to the experimental models for these cases. However, some differences between the models exist. There is a time lag after the initial compression response between the finite element analyses versus the experiments. It is believed this time lag to be due to the lack of the interface in the computational models. The interface could cause the backfilled center material to deform out of phase with the motion of the remainder of the specimen block. The experimental displacement data show secondary oscillations at times 0.14 seconds and 0.22 seconds for the 3 Joule case, while for the 5 Joule case, these oscillations occur at 0.13 seconds and 0.22 seconds. The finite element model does not exhibit these secondary oscillations in the 3 Joule case. In the 5 Joule case, the finite element model shows the initial development of these oscillations with a delayed formation at 0.25 seconds.

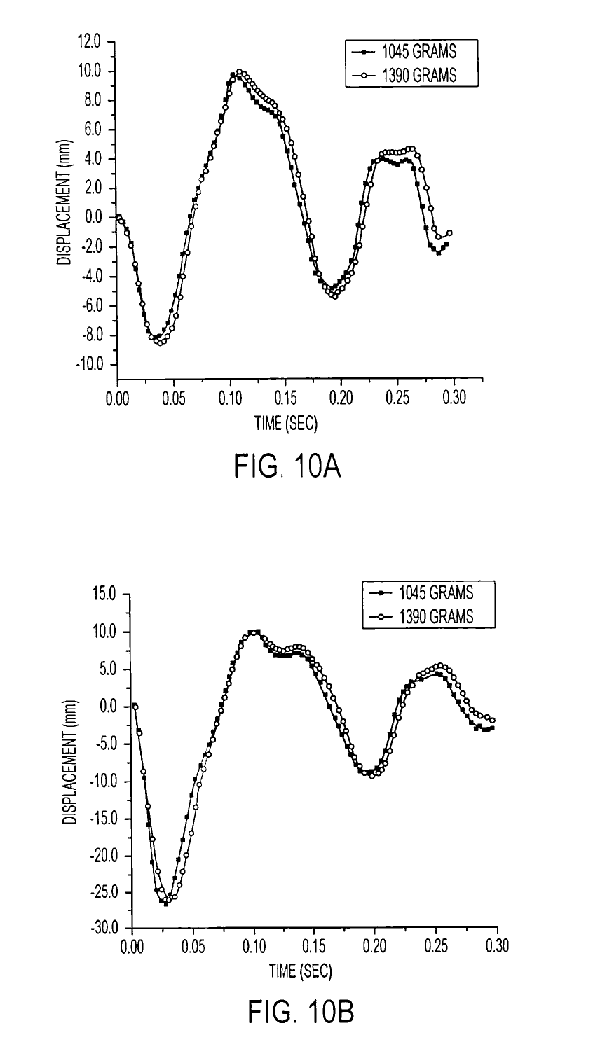

FIG. 10 compares the predicted displacement responses of the specimen using 1045 and 1390 gram balls with an impact energy of 5 Joules at the center of the specimen (FIG. 10a) and at 51 mm from the impact surface (FIG. 10b). Both figures show only minor deviations in displacements for the different ball sizes. This indicates that for these type ball sizes there is a negligible effect on displacements.

The Fast Fourier Transforms of the finite element and the experimental displacement data at the center of the specimen block for the 3 and 5 Joule, 1045 gram ball cases are shown in Table 2.

TABLE 2
Fast Fourier Transforms of finite element and experimental
displacement data at center, 1045 gram ball.

| Resonance Peak | Computational Model (Hz) | Experimental Model (Hz) |
|---|---|---|
| | 3 Joules | |
| 1 | 9.89 | 9.57 |
| 2 | — | 14.85 |
| 3 | 21.36 | 22.92 |
| 4 | 32.53 | 36.25 |
| | 5 Joules | |
| 1 | 7.7 | 9.25 |
| 2 | — | 15.18 |
| 3 | 22.29 | 22.90 |

The finite element and experimental frequencies match fairly well. An additional frequency peak is observed in both cases of the experimental model which is not observed in the finite element model. These additional frequencies are most likely caused by the interface between the block gelatin and embedded cylinder gelatin of the cylindrical hole and indicance of including these interface in future computational models.

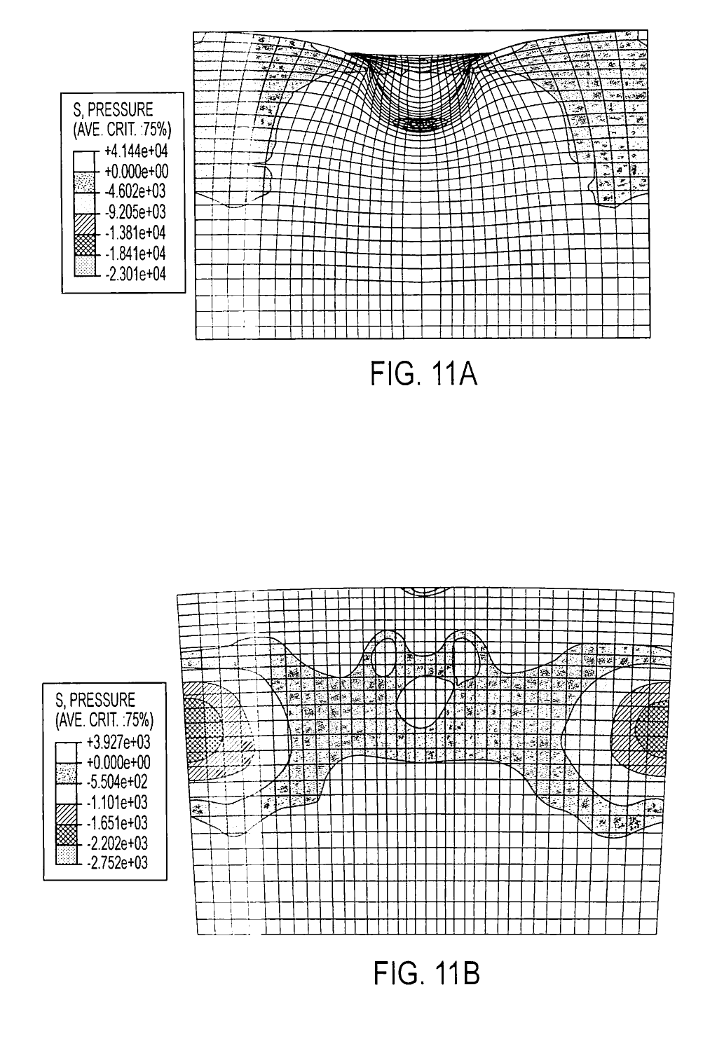

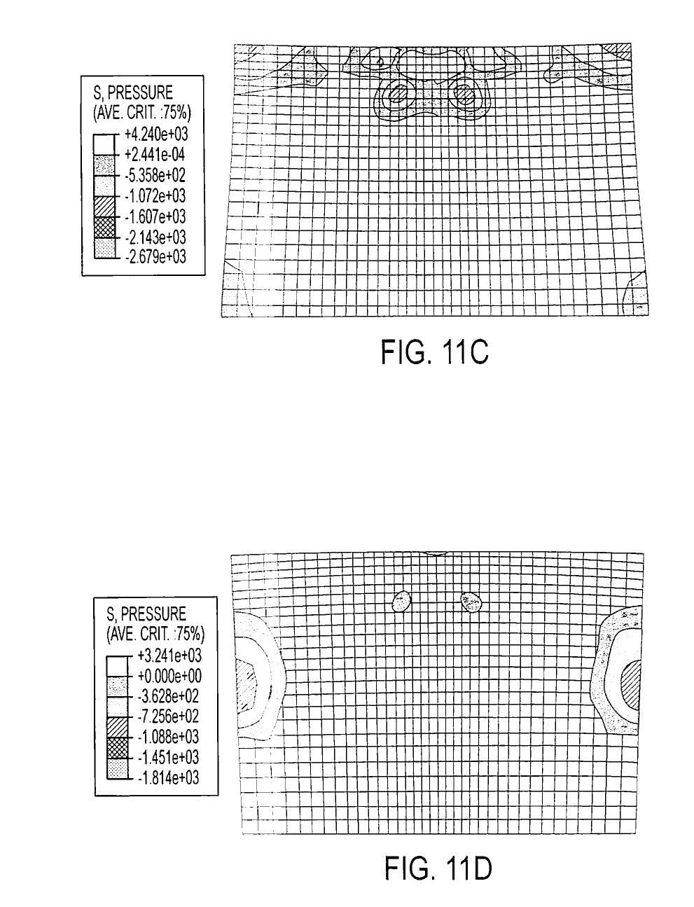

Pressure contour plots of the 10 Joule, 1390 gram ball drop case for times of 0.027, 0.066, 0.076, and 0.081 seconds are shown in FIG. 11, a-d, respectively. The contour values have been selected to emphasize the negative regime. Previous research has shown that negative pressures causes more blunt trauma damage to biological tissue than positive pressures. FIG. 11a is the pressure contour at the point of maximum sphere indentation. A high pressure area forms immediately below the impact region. FIG. 11b shows the formation of two symmetric pressure regions at approximately 40 mm from the impact surface. As the ball rebounds from the impact surface (FIG. 11c), the block begins to lift off the surface of the table and pressure zones at the sides develop. FIG. 11d shows a negative pressure region as it expands from the base of the block to the impact surface with the maximum pressure occurring along the sides of the block.

### EXAMPLE 2

### Thoracic Surrogate Tests

Impact experiments: Experimental and finite-element analysis was applied to study the dynamic response of the thoracic surrogate model to low velocity impacts. The experiment was designed to relate impactor velocity, size, and associated frequencies to material damage. The experiment measured displacements, wave propagations, and stress distribution by using embedded accelerometers, pressure sensors, and photoelastic techniques. These measurements provided the basis for understanding the thoracic surrogate model response to subsequent exposures to vibrations, shock tube pressure pulses, and blasts. A thoracic surrogate model consisting of cylindrical lungs and surrounding tissue was constructed. The computational model predicted surface and internal displacements as well as stress field distributions similar to those found in experimental drop tests using photoelastic visualization techniques. This computational model was used for developing and understanding more complex thoracic models.

Shaker Table and Shock Tube Experiments: A vibrational shaker was used to perform tests on a thoracic surrogate with constant peak accelerations of 0.25, 0.50, and 1.00 g, and frequencies from 5 Hz to 1 kHz. In the frequency domain, resonance peaks of acceleration and pressures for the critical components of the complex system were measured for the first four modes of vibrations. The thoracic surrogate was also subjected to dynamic pressure pulse conditions using a shock tube facility.

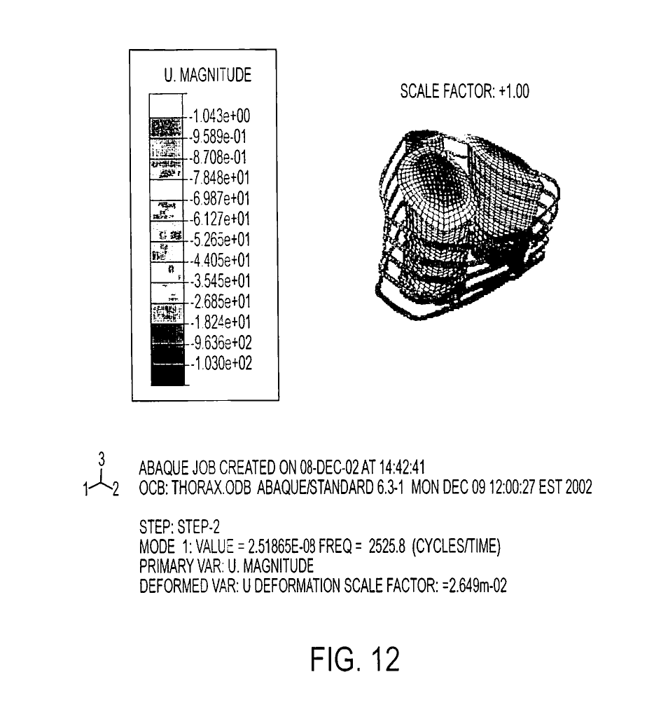

A steady-state modal analysis of the thoracic surrogate model, as shown in FIG. 12, was performed to analyze the mechanical vibration characteristics of the thorax. This compared well with measured responses. This model provided local stress, strain, pressure, and acceleration data that were used to develop a damage criterion and optimize sensor locations for the thoracic surrogate model.

Finite element methods were applied to study the dynamics of human tissue simulant materials and compared to experimental tests. The computational displacement and frequency histories compared well to the experimental models. The computational predictions also provided pressure contours from ball impacts on tissue simulants. The interface present in the experimental model is unaccounted for in the existing finite element simulations. The lack of this in-e may be the leading source of discrepancy in the results. The understanding of these interface properties will be very important in finite element modeling of the more complex Thoracic surrogate model.

The thoracic surrogate model has been tested at various blast test sites to measure free field and confined-space responses to blast. The information from these tests and the shock tube tests will be used to develop transfer functions relating blast waves to the body response. During one of the free field test series, the thoracic surrogate model was used to evaluate the effectiveness of body armor against blast. Body armor is a soft vest used with and without hard ceramics inserts. These types of body armor systems are designed to provide effective protection against ballistic weapons threat. The free field blast data, in conjunction with shock tube tests, showed that the soft and combined soft and hard body armor systems do, however, slightly increase the peak lung pressures when subjected to blast. This suggests that future body armor designs should attempt to protect against blast as well as to preserve the ballistic protection capabilities. The thoracic surrogate model can be used to guide and evaluate new body armor designs by linking the external ballistic and pressure loads to the internal body dynamics.

Obviously, many modifications and variations of the present invention are possible in light of the above teachings. It is therefore to be understood that, within the scope of the appended claims, the invention may be practiced otherwise than as specifically described.

What is claimed as new and desired to be protected by Letters Patent of the United States is:

1. A method for making simulated human tissue comprising the steps of:
Combining ordinance gelatin, chilled water and an antimicrobial agent in a desired volume or weight percentage;
Hydrating said mixture;
Heating and stirring said mixture;
Pouring said mixture into a container; and
Chilling said mixture until the simulated tissue is set.

2. The method of claim 1 wherein said ordinance gelatin is TYPE 250A Gelatin.

3. The method of claim 1 wherein said water is chilled to 7-10° C.

4. The method of claim 1 wherein said antimicrobial agent is Cinnamon 100% pure leaf oil.

5. The method of claim 1 wherein said volume or weight percentage ranges from about 15% to about 25%.

6. The method of claim 5 wherein said volume or weight percentage is more preferably about 20%.

7. The method of claim 1 wherein said hydrating step is at least about 2.5 hours.

8. The method of claim 7 wherein said hydrating step more preferably is at least 5 hours.

9. The method of claim 1 wherein said heating and stirring step starts from approximately room temperature increasing by increments of 20° F. up to about 140° F.

10. The method of claim 9 wherein said heating and stirring step continues until the mixture is clear.

11. The method of claim 1 wherein said chilling step occurs at a temperature from about 7° C. to about 10° C.

12. A method of making simulated lung tissue comprising the steps of:
combining ordinance gelatin, chilled water and an antimicrobial agent in a desired volume or weight percentage;
hydrating said mixture;
heating and stirring said mixture in a fume hood;
adding microspheres to said mixture;
stirring said mixture containing the microspheres;
pouring said mixture into a container; and
chilling said mixture until the simulated lung tissue is set.

13. The method of claim 12 wherein said ordinance gelatin is TYPE 250A Gelatin.

14. The method of claim 12 wherein said water is chilled to 7-10° C.

15. The method of claim 12 wherein said antimicrobial agent is Cinnamon leaf oil.

16. The method of claim 12 wherein said volume or weight percentage ranges from about 15% to about 25%.

17. The method of claim 16 wherein said volume or weight percentage is more preferably about 20%.

18. The method of claim 12 wherein said hydrating step is at least about 2.5 hours.

19. The method of claim 18 wherein said hydrating step more preferably is at least 5 hours.

20. The method of claim 12 wherein said heating and stirring step starts from approximately room temperature increasing by increments of 20° F. up to about 140° F.

21. The method of claim 20 wherein said heating and stirring step continues until the mixture is clear.

22. The method of claim 12 wherein said microspheres are Expancel DE microspheres.

23. The method of claim 22 wherein a range of from about 0.30 grams to about 0.60 grams of microspheres per 3500 ml mixture are added to the mixture.

24. The method of claim 22 wherein most preferably, about 0.42 grams of microspheres per 3500 ml mixture are most preferably added to the mixture.

25. The method of claim 12 wherein said second stirring step preferably creates a vortex in the mixture.

26. The method of claim 12 wherein said container approximates the shape of a lung.

27. The method of claim 12 wherein said chilling step occurs at a temperature from about 7° C. to about 10° C.

28. A modified ballistic gelatin material for use as simulated tissue comprised of ordinance gelatin, chilled water and an antimicrobial agent, wherein said ordinance gelatin is added in a desired volume or weight percentage.

29. A simulated lung comprised of modified ballistic gelatin and microspheres.

30. An instrumented thoracic model comprised of:
simulated skeletal components, including, but not limited to, anatomically correct spine, rib cage, and sternum;
simulated tissue surrounding said simulated skeletal component;
simulated organs, including but not limited to, a heart and two lungs, enclosed in said simulated tissue; and
sensors, optimally placed in said simulated tissue, said simulated organs and said simulated skeletal components for measuring the effects of impacts on said thoracic model.

31. The instrumented thoracic model of claim 30, wherein said simulated tissue is comprised of modified ballistic gelatin material.

32. The instrumented thoracic model of claim 30, wherein said simulated lung is comprised of modified ballistic gelatin and microspheres.

33. The instrumented thoracic model of claim 30, wherein said sensors are optimally placed by Applying PCA analysis to the heart, lungs and spine by
taking the normalized displacements of all the primary modes;
calculating the principal components from the normalized displacements;
selecting only the principal components which have a significant influence on the system;
using the major principal components on the normalized displacement data;
reevaluating the reduced data set for patterns, relationships; and
grouping/clustering for placement of the sensors.

34. A method of optimizing the placement of sensors in a thoracic model, comprising the steps of:
Applying PCA analysis to the heart, lungs and spine by taking the normalized displacements of all the primary modes;
calculating the principal components from the normalized displacements;
selecting only the principal components which have a significant influence on the system;
using the major principal components on the normalized displacement data;
reevaluating the reduced data set for patterns, relationships; and
grouping/clustering for placement of the sensors.
